(19) United States
(12) Patent Application Publication
(10) Pub. No.: US 2014/0084206 A1
(43) Pub. Date: Mar. 27, 2014

(54) Treating Additives for the Deactivation of Sulfur Species Within a Stream

(71) Applicant: BAKER HUGHES INCORPORATED, Houston, TX (US)

(72) Inventors: Jianzhong Yang, Missouri City, TX (US); James Michael Brown, Lago Vista, TX (US)

(73) Assignee: BAKER HUGHES INCORPORATED, Houston, TX (US)

(21) Appl. No.: 13/628,224

(22) Filed: Sep. 27, 2012

(51) Int. Cl. C07C 217/40; C02F 1/68; C09K 3/00; C10G 29/20; C10G 29/04

(52) U.S. Cl. 252/180; 208/236; 208/230; 564/294; 564/291; 252/189; 252/182.12; 252/182.3; 210/749

(57) ABSTRACT

There is provided, in one form, a method for at least partially deactivating a sulfur species from a stream, such as but not limited to a hydrocarbon stream, an aqueous stream, and mixtures thereof. A treating mixture may be introduced into the stream in an amount effective to at least partially deactivate the sulfur species from the stream. The treating mixture may include a compound having the general formula:

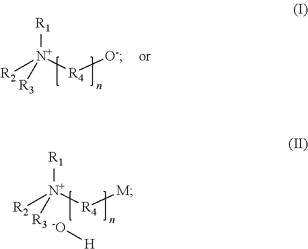

and combinations thereof. R₁ may be a C₁-C₄ hydrocarbyl group. R₂ may be a C₁-C₄ hydrocarbyl group that is the same or different as R₁. R₃ may be an ethanol or an isopropanol moiety. R₄ may be an ethylene oxide moiety, propylene oxide moiety, butylene oxide moiety, and combinations thereof. n may be an integer from 1 to 100. M may be a hydrogen or a metal ion.

### TECHNICAL FIELD

The present invention relates to methods and compositions for deactivating sulfur species within a stream, and more particularly relates, in one non-limiting embodiment, to introducing a treating additive into a hydrocarbon stream, an aqueous stream, and mixtures thereof in an amount effective to at least partially deactivate the sulfur species within the stream.

### BACKGROUND

The presence of sulfur species in hydrocarbon fluids and aqueous streams is undesirable for various reasons. The subterranean reservoirs currently being developed have increased amounts of sulfur species within the produced hydrocarbon streams (oil and gas). Hydrogen sulfide and mercaptans are life threatening reactive species that are often associated with obnoxious odors. Due to their acidity, H₂S and small mercaptans are very corrosive to the oil well and surface equipment during storage and transportation.

During combustion, sulfur-rich hydrocarbon streams also produce heavy environmental pollution. When sulfur-rich streams contact metals, sulfur species lead to brittleness in carbon steels and to stress corrosion cracking in more highly alloyed materials. Moreover, hydrogen sulfide and mercaptans, in various hydrocarbon or aqueous streams pose a safety hazard and a corrosion hazard. A quick removal of these odorous and environmental malicious species would be desirable in both oilfield and refinery operations.

For the reasons mentioned, attempts have been made to wash out, or chemically convert, the sulfur species from hydrocarbon fluids and aqueous systems. Sour gas is a natural gas or any other gas containing significant amounts of hydrogen sulfide (H₂S). There are several classes of scavengers available for deactivating or removing sulfur species from a hydrocarbon or aqueous stream, but many of them have serious limitations.

For example, nitrogen-containing hydrogen sulfide scavengers, such as hydrotriazine-based additives, have been in the industry for quite some time. However, the amines released while scavenging the sulfur species pose an overhead corrosion threat in various downstream processes, including distillation columns. Formaldehyde is a nitrogen-free scavenger, but it is also a potential carcinogen. Glyoxal is another nitrogen-free hydrogen sulfide scavenger, but its application is often limited due to its corrosivity and low boiling point. Metal oxides have also been proposed, but such applications are narrowed by the handling challenges and solid residual formation concerns to downstream refining catalysts and processes. Acrolein is a clean and extremely potent hydrogen sulfide/mercaptan scavenger, but it requires special handling due to toxicity concerns.

Trialkyl ethanol ammonium hydroxide is an organic base that has been a useful sulfur scavenger, particularly with mercaptans, but its usage is limited because of its ability to self-assemble with both water and small alcohol molecules. This self assembly causes a reaction that forms a hydrate gel structure, which is more likely to occur in the presence of both oil and water.

Thus, it would be desirable if a sulfur species scavenger could be discovered for deactivating sulfur species, but that is compatible with sulfur scavengers found in water-based fluids, oil-based fluids, and combinations thereof.

### SUMMARY

There is provided, in one form, a method for at least partially deactivating a sulfur species from a stream, such as but not limited to a hydrocarbon stream, an aqueous stream, and mixtures thereof. A treating additive may be introduced into the stream in an amount effective to at least partially deactivate the sulfur species from the stream. The treating additive may include a compound having the general formula:

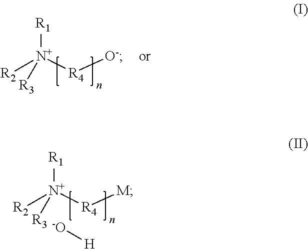

and
combinations thereof.

R₁ may be a C₁-C₄ hydrocarbyl group. R₂ may be a C₁-C₄ hydrocarbyl group that is the same or different as R₁. R₃ may be an ethanol moiety, or an isopropanol moiety. R₄ may be an ethylene oxide moiety, propylene oxide moiety, butylene oxide moiety, and combinations thereof. n may be an integer from 1 to 100. M may be a hydrogen or a metal ion.

In another non-limiting embodiment, the amount of the treating additive within the stream may range from about 1 ppm to about 50,000 ppm, and the compound may have the general formula:

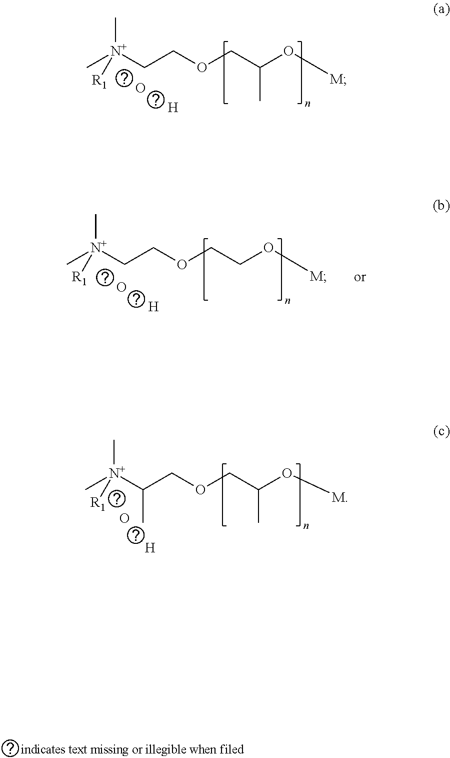

There is provided, in another embodiment, a treated stream having a treating additive therein for at least partially deactivating a sulfur species within the stream, such as a hydrocarbon stream, an aqueous stream, and mixtures thereof. The treating additive may include compounds (I), (II), and combinations thereof.

The compounds appear to react with the sulfur species to at least partially render the sulfur species from further reactions within the stream.

### BRIEF DESCRIPTION OF THE DRAWINGS

FIG. 1 is a graph illustrating the decreased amounts of various sulfur species present in a bitumen diluent fluid after treatment with a sulfur scavenger.

### DETAILED DESCRIPTION

It has been discovered that a treating additive may be introduced into a stream in an effective amount to at least partially deactivate the sulfur species therefrom. The stream may be or include, but is not limited to a hydrocarbon stream, an aqueous stream, and mixtures thereof. By “deactivate” the sulfur species, it is meant that the sulfur species reacts with the compound(s) of the treating additive to at least partially render the sulfur species incapable from further reactions within the stream. Alternatively, “deactivate” may be binding up the sulfur species so that it does not become problematic to downstream operations. In one non-limiting embodiment, the deactivated sulfur species may be physically removed from the stream.

The compounds within the treating additive are organic bases that may act as sulfide scavengers to control the amount of active sulfur species within the stream. These compounds, described in more detail below, are less likely to form a hydrate gel structure even in the presence of both water-based fluids and oil-based fluids. The compounds may also act as a pH control additive by increasing the pH of the fluid through a neutralization reaction.

“Treating additive” is defined herein to include at least one compound of the general formula:

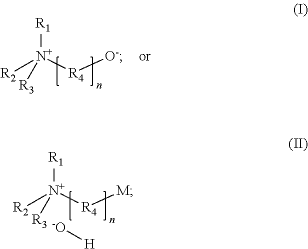

and
combinations thereof.
R₁ may be a C₁-C₄ hydrocarbyl group.
R₂ may be a C₁-C₄ hydrocarbyl group that is the same or different as R₁.
R₃ may be an ethanol moiety, or an isopropanol moiety.
R₄ may be an ethylene oxide moiety, propylene oxide moiety, butylene oxide moiety, and combinations thereof.
n may be an integer from 1 to 100, and
M is a hydrogen or a metal ion. The metal ion may be or include, but is not limited to, Li, Na, K, and combinations thereof.

In some instances, the treating additive may include a distribution of compounds (I) and/or (II) having the same or different n values. In one non-limiting embodiment, the treating additive may have only one specific compound with a particular n value. Compound (II) may include compounds with the general formula:

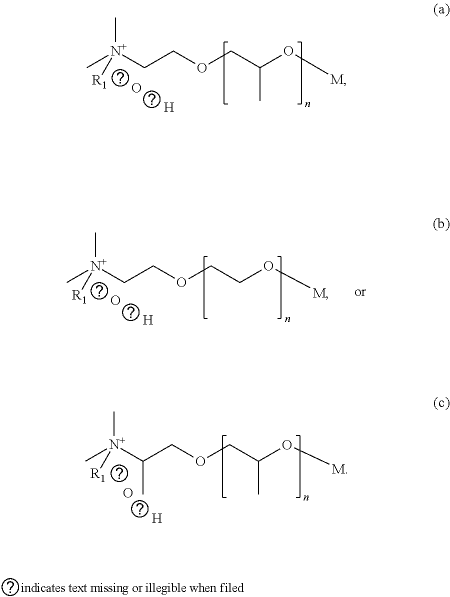

The treating additive may include compound (I), compound (II), and combinations thereof. It is difficult to predict with precision the dosage of the treating additive required to deactivate H₂S, mercaptans and/or polysulfides in a stream. An optimum application level will depend on a number of factors, including, but not limited to, the nature of any hydrocarbons in the stream; the level of H₂S, mercaptans, and/or polysulfides; the nature of the mercaptans and their proportions; the temperature of the stream; the particular nature of the compounds in the treating additive, etc. ‘An amount effective to deactivate the sulfur species’ is defined herein to mean any amount of the treating additive to at least partially deactivate the sulfur species and render the sulfur species from further reactions within the stream, or at least bind up the sulfur species so that it does not become problematic to downstream operations.

However, to give a sense of appropriate treating levels, the amount of the compound within the stream may range from about 1 ppm independently to about 50,000 ppm, alternatively from about 5 ppm independently to about 5,000 ppm. The amount of the treating additive within the stream may range from about 1 ppm independently to about 100,000 ppm, alternatively from about 10 ppm independently to about 10,000 ppm. As used herein with respect to a range, “independently” means that any lower threshold may be used together with any upper threshold to give a suitable alternative range.

Complete deactivation and/or removal of the sulfur species from the stream is desirable, but it should be appreciated that complete deactivation and/or removal is not necessary for the methods discussed herein to be considered effective. Success is obtained if more sulfur species are deactivated using the treating additive than in the absence of the treating additive. Alternatively, the methods described are considered successful if a majority of the sulfur species is deactivated.

The sulfur species to be deactivated within the stream may be or include, but is not limited to hydrogen sulfide, mercaptans, polysulfides, and combinations thereof. A mercaptan may be any sulfur-containing compound of the general formula R—SH, such as ethyl mercaptan/ethanethiol. The use of the term ‘polysulfide’ herein generally refers to a class of sulfur species with alternating chains of sulfur atoms and hydrocarbons, such as —[(CH₂)ₘ—Sₓ]ₙ—.

The treating additive may be part of a treating mixture that may include a solvent, such as but not limited to an aromatic solvent, an alcohol based solvent, an ester based solvent, an amide based solvent, and combinations thereof. The aromatic solvent may be or include, but is not limited to, Aromatic 100, Aromatic 150, kerosene, diesel, or mixtures thereof. The concentration of the solvent within the treating mixture may be from about 1 wt % independently to about 99 wt %, alternatively from about 5 wt % independently to about 80 wt %. The concentration of the compound within the treating mixture may be from about 1 to about 99 wt % of the treating mixture, alternatively from about 20 wt % independently to about 80 wt %. The treating mixture may also include other additives, such as but not limited to a corrosion inhibitor, a dehazer, a conductivity improver, a foamer, a demulsifer, and combinations thereof.

The treating mixture may be used in conventional “in-line” injection systems and injected at any point in-line suitable to allow the mixture to react with the gaseous or liquid stream, e.g. at the well-head, separators, etc. The treating additive may also be used in conventional scrubber tower systems. Other applications of the treating additives described herein in other conventional systems or systems to be developed will become apparent to those skilled in the art.

Examples which are not meant to limit the invention, but rather to further illustrate the various embodiments.

### PREPARATION EXAMPLES 1-7

### Example 1

Preparation of 1964-271: In a typical reaction, dimethyl ethanolamine (118.0 g) and aromatic solvent (72 g) were charged into a stainless steel par reactor. The mixture was oxyalkylated with propylene oxides (PO) until about 3.0 moles of PO were added, and the reaction slowed down. Deionized (DI) water (23.9 g) was added to the mixture in one batch. The mixture was ethoxylated with ethylene oxide until all water was consumed. The reaction was monitored by using the Karl-Fisher titration method to monitor the amounts of water until such water content was less than 0.1 vol %. The resulting product was scavenger 1964-271 noted in Table 2.

### Example 2

Preparation of 1964-351: Dimethyl ethanolamine (178.0 g) and methanol (32.0 g) were charged into a stainless steel par reactor. The mixture was oxyalkylated with propylene oxide (PO) until about 3.1 moles of PO were gradually added, and the reaction slowed down. DI water (36.0 g) was added to the mixture in one batch. The mixture was ethoxylated with ethylene oxide until all water was consumed. The reaction was monitored by Karl-Fisher until the water content was less than 0.1 vol %. The resulting product was scavenger 1964-351 noted in Table 2.

### Example 3

Preparation of 1964-371: Dimethyl isopropanolamine (154.8 g) and isopropanol (60.0 g) were charged into a stainless steel par reactor. The mixture was oxyalkylated with propylene oxides (PO) until about 3.0 moles of PO were added, and the reaction slowed down. DI water (36.0 g) was added to the mixture in one batch. The mixture was ethoxylated with ethylene oxide until all water was consumed. The reaction was monitored by Karl-Fisher until the water content was less than 0.1 vol %. The resulting product was scavenger 1964-371 noted in Table 2.

### Example 4

Preparation of 1964-411: Dimethyl isopropanolamine (206.0 g) and water (36.0 g) were charged into a stainless steel par reactor. The mixture was oxyalkylated with ethylene oxide (7.0 g) and propylene oxide (108.0 g) until the water content was less than 0.1 vol % as monitored by Karl-Fisher. The resulting product was marked as scavenger 1964-411 noted in Table 5.

### Example 5

Preparation of 1964-491: Dimethyl ethanolamine (90.0 g), isopropanol (10.0 g) and aromatic 100 (100.0 g) were charged into a stainless steel par reactor. The mixture was oxyalkylated with propylene oxide until about 2.5 moles of PO were gradually consumed, and the reaction slowed down. DI water (18.2 g) was added to the mixture in one batch. The mixture was ethoxylated with ethylene oxide until all water was consumed. The reaction was monitored by Karl-Fisher until the water content was less than 0.1 vol %. The resulting product was marked as scavenger 1964-491 noted in Table 2.

### Example 6

Preparation of 1964-591: Dimethyl ethanolamine (135.0 g), isopropanol (60.0 g), aromatic 100 (100.0 g) and 2,6-di-t-butyl phenol (0.1 g) were charged into a stainless steel par reactor. The mixture was oxyalkylated with propylene oxide (233.7 g) until about 4.0 moles of PO were gradually added, and the reaction slowed down. DI water (27.0 g) was added to the mixture in one batch. The mixture was ethoxylated with ethylene oxide until all water was consumed. The reaction was monitored by Karl-Fisher until the water content was less than 0.1 vol %. The resulting product was marked as scavenger 1964-591 noted in Table 2.

### Example 7

Preparation of 1964-272: Scavenger 1964-272, noted in Table 1, Table 3 and Table 4 was formulated by mixing 95 vol % of 1964-271 with 5 vol % of isobutanol.

### SCAVENGING EXAMPLES

### Tables 1-5

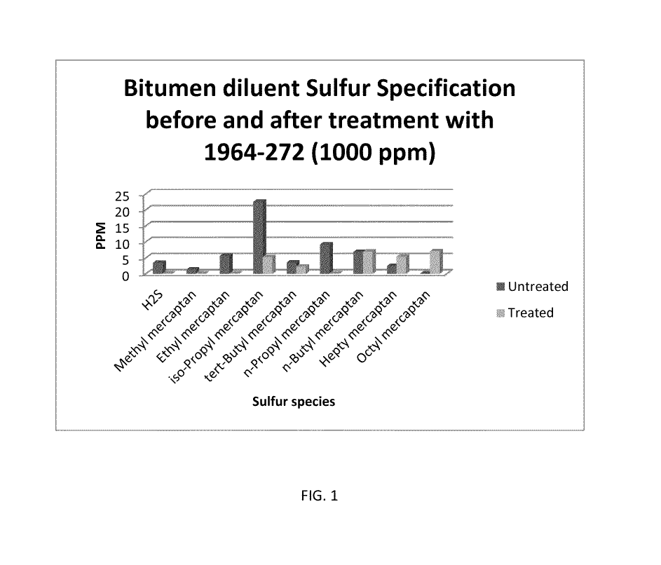

FIG. 1 is a graph illustrating the decreased amount of sulfur species present in a bitumen diluent fluid after treatment with a sulfur scavenger compound; the results are also noted in Table 1. The bitumen diluent was treated with 1000 ppm of 1964-272. The levels of various mercaptans from the bitumen diluent were tested before and after the scavengers were mixed therein. As noted by the graph and Table 1, the treating additive appears to be effective against most mercaptans.

TABLE 1
Sulfur Specification analysis on the diluents sample by GC-MS
(ASTM D5623)

| | | Diluent sample |
|---|---|---|
| | Diluent Sample Sulfur | treated with 1964-272 |
| Sulfur Specification | species content (ppm) | (1000 ppm) |
| H2S | 3.4 | 0.1 |
| Methyl mercaptan | 1.3 | 0.1 |
| Ethyl mercaptan | 5.6 | 0.1 |
| iso-Propyl mercaptan | 22.4 | 5.2 |
| tert-Butyl mercaptan | 3.5 | 2.2 |
| n-Propyl mercaptan | 9.1 | 0.1 |
| n-Butyl mercaptan | 6.8 | 6.9 |
| Hepty mercaptan | 2.4 | 5.3 |
| Octyl mercaptan | 0.1 | 7 |
| Total mercaptan | 50 | 27 |

The fluids tested in Tables 1-4 were treated with various scavengers where each scavenger was mixed with a particular fluid and shaken for 20 seconds. The scavenger tested with each fluid is noted within the Tables. The reactions to form the scavengers are described in Preparation Examples 1-7 above. The data from Table 1 is discussed above. The data in Table 2 was generated using a propane thiol artificially dosed Aromatic-100 fluid; the data from Table 3 was generated using a natural gas liquid (NGL) sample containing mercaptans; the data from Table 4 was generated using a bitumen diluent containing H2S and mercaptans. The samples were placed at room temperature overnight unless otherwise noted. The sulfur content was determined by AgNO₃ potentiometry titration by using the guidelines of ASTM D3227 (U0P163). Sulfur specification was determined using ASTM D5623.

Table 2 indicates the decreased amount of Mercaptan S within the propane thiol artificially dosed aromatic-100 fluid after treatment with each scavenger. Table 3 indicates the decreased amount of Mercaptan S within the NGL sample containing mercaptans after treatment with scavenger 1964-272. Table 4 indicates the decreased amounts of H₂S and Mercaptan S within the bitumen diluent after treatment with scavenger 1964-272.

TABLE 2
Propane thiol artificially dosed aromatic-100 fluid was treated with the
indicated scavenger by ASTM D3227

| Scavenger | Scavenger | Tested fluid w/ | Mercaptan S |
|---|---|---|---|
| # | Dosage (ppm) | 1-Propanthiol | (ppm) |
| Blank | — | Aromatic 100 | 140 |
| 1964-271 | 2000 | Aromatic 100 | ND |
| 1964-271 | 4000 | Aromatic 100 | ND |
| 1964-351 | 1000 | Aromatic 100 | 50.4 |
| 1964-351 | 2000 | Aromatic 100 | ND |
| 1964-351 | 4000 | Aromatic 100 | ND |
| 1964-371 | 1000 | Aromatic 100 | 65.0 |
| 1964-371 | 2000 | Aromatic 100 | ND |
| 1964-491 | 2000 | Aromatic 100 | 118.6 |
| 1964-491 | 4000 | Aromatic 100 | 45.3 |
| 1964-591 | 2000 | Aromatic 100 | ND |
| Blank | — | Aromatic 100 | 140 |

TABLE 3
A natural gas liquid (NGL) sample containing mercaptans was treated
with the indicated scavenger by ASTM D3227

| | Scavenger | | Mercaptan S |
|---|---|---|---|
| Scavenger # | Dosage (ppm) | Tested fluid | (ppm) |
| Blank | — | NGL | 748 |
| 1964-272 | 3000 | NGL | 467 |

TABLE 4
A bitumen diluent containing H₂S and mercaptans was treated
with the indicated scavenger by titration (D5623)

| | Scavenger | | | Mercaptan S |
|---|---|---|---|---|
| Scavenger # | Dosage (ppm) | Tested fluid | H₂S | (ppm) |
| Blank | — | Condensate | 3.4 | 50 |
| 1964-272 | 1000 | Condensate | 0.1 | 27 |

TABLE 5
A naphtha sample containing mercaptans was treated with
the indicated scavenger by ASTM D3227

| | Scavenger | | Mercaptan S |
|---|---|---|---|
| Scavenger # | Dosage (ppm) | Tested fluid | (ppm) |
| Blank | — | Naphtha | 550 |
| 1964-411 | 4000 | Naphtha | 467 |

In the foregoing specification, the invention has been described with reference to specific embodiments thereof, and has been described as effective in providing methods and compositions for introducing a treating additive into a stream for at least partially deactivating the sulfur species therein. It is to be understood that the invention is not limited to the exact details of reaction conditions, proportions, etc. shown and described, as modifications and equivalents will be apparent to one skilled in the art. Accordingly, the invention is therefore to be limited only by the scope of the appended claims. Further, the specification is to be regarded as an illustrative, rather than a restrictive sense. For example, specific amounts of sulfur scavenger compounds, solvents, reactant proportions, reaction conditions, dosages and the like, falling within the claimed parameters but not specifically identified or tried in a particular method, are anticipated to be within the scope of this invention.

The present invention may suitably comprise, consist or consist essentially of the elements disclosed and may be practiced in the absence of an element not disclosed. For instance, the method may consist of or consist essentially of removing a sulfur species from a stream, such as but not limited to, a hydrocarbon stream, an aqueous stream, and mixtures thereof by introducing a treating additive into the stream in an amount effective to at least partially deactivate the sulfur species therein where the treating mixture includes at least compound (I), compound (II), and/or combinations thereof. The composition may include a treating additive for at least partially deactivating a sulfur species from a stream, such as a hydrocarbon stream, an aqueous stream, and/or mixtures thereof where the treating additive consists of or consists essentially of compound (I), compound (II), and/or combinations thereof.

The words “comprising” and “comprises” as used throughout the claims, are to be interpreted to mean “including but not limited to” and “includes but not limited to”, respectively.

What is claimed is:

1. A method for at least partially deactivating a sulfur species from a stream selected from the group consisting of a hydrocarbon stream, an aqueous stream, and mixtures thereof; wherein the method comprises introducing a treating additive into the stream in an amount effective to at least partially deactivate the sulfur species from the stream; and wherein the treating additive comprises a compound of the general formula selected from the group consisting of: 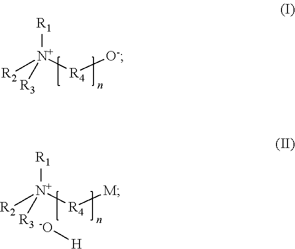
and
combinations thereof; and
wherein:
R₁ is a C₁-C₄ hydrocarbyl group,
R₂ is a C₁-C₄ hydrocarbyl group that is the same or different as R₁,
R₃ is an ethanol or an isopropanol moiety,
R₄ is an ethylene oxide moiety, propylene oxide moiety, butylene oxide moiety, and combinations thereof,
n is an integer from 1 to 100, and
M is a hydrogen or a metal ion.

2. The method of claim 1, wherein the compound (II) is selected from the group consisting of: 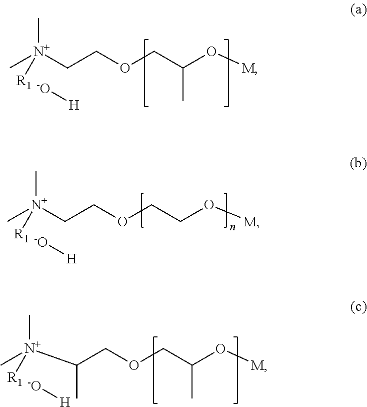
and
combinations thereof.

3. The method of claim 1, wherein the treating additive comprises a solvent.

4. The method of claim 3, wherein the concentration of the solvent within the treating mixture ranges from about 1 wt % to about 99 wt %.

5. The method of claim 1, wherein the amount of the treating additive within the stream ranges from about 1 ppm to about 50,000 ppm.

6. The method of claim 1, wherein the sulfur species is selected from the group consisting of hydrogen sulfide, mercaptans, polysulfides, and combinations thereof.

7. The method of claim 1, wherein the metal ion is selected from the group consisting of Li, Na, K, and combinations thereof.

8. A method for deactivating a sulfur species from a stream selected from the group consisting of a hydrocarbon stream, an aqueous stream, and mixtures thereof; wherein the method comprises introducing a treating additive into a stream in an amount effective to at least partially deactivate the sulfur species from the stream; and wherein the treating additive comprises a compound of the general formula selected from the group consisting of: 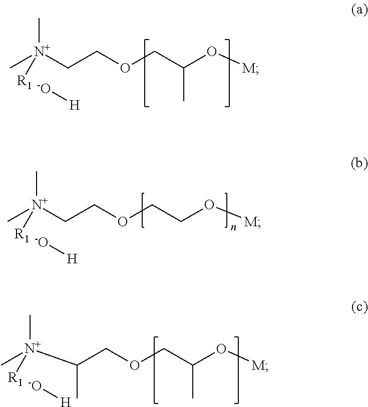
and
combinations thereof; and
wherein:
wherein R₁ is a C₁-C₄ hydrocarbyl group,
wherein n is an integer from 1 to 100, and
wherein M is a hydrogen, or a metal ion selected from the group consisting of Li, Na, K, and combinations thereof; and
wherein the amount of the treating additive within the stream ranges from about 1 ppm to about 50,000 ppm.

9. A treating additive for deactivating a sulfur species from a stream selected from the group consisting of a hydrocarbon stream, an aqueous stream, and mixtures thereof; and wherein the treating additive comprises a compound of the general formula selected from the group consisting of: 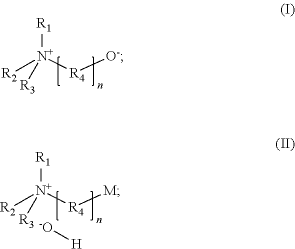
and
combinations thereof; and
wherein:
R₁ is a C₁-C₄ hydrocarbyl group,
R₂ is a C₁-C₄ hydrocarbyl group that is the same or different as R₁,
R₃ is an ethanol or an isopropanol moiety,
R₄ is an ethylene oxide moiety, propylene oxide moiety, butylene oxide moiety, and combinations thereof,
n is an integer from 1 to 100, and
M is a hydrogen or a metal ion.

10. The treating additive of claim 9, wherein the compound (II) is selected from the group consisting of: 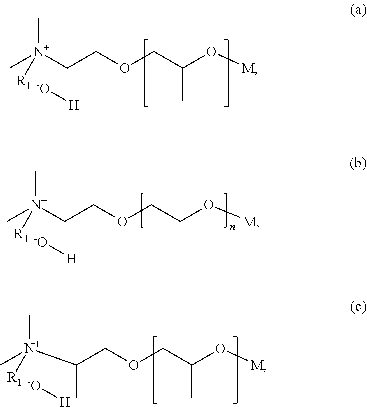
and
combinations thereof.

11. The treating additive of claim 9, wherein the treating additive comprises a solvent.

12. The treating additive of claim 11, wherein the concentration of the solvent ranges from about 1 wt % to about 99 wt %.

13. The treating additive of claim 11, wherein the solvent is selected from the group consisting of an aromatic solvent, an alcohol based solvent, an ester based solvent, an amide based solvent, and combinations thereof.

14. The treating additive of claim 9, wherein the amount of the treating additive within the stream ranges from about 1 ppm to about 50,000 ppm.

15. The treating additive of claim 9, wherein the sulfur species is selected from the group consisting of hydrogen sulfide, mercaptans, polysulfides, and combinations thereof.

16. The treating additive of claim 9, wherein the metal ion is selected from the group consisting of Li, Na, K, and combinations thereof.

17. A treated stream having a treating additive therein for at least partially deactivating a sulfur species from a stream selected from the group consisting of a hydrocarbon stream, an aqueous stream, and mixtures thereof; wherein the treated stream comprises a treating additive having a compound of the general formula selected from the group consisting of: 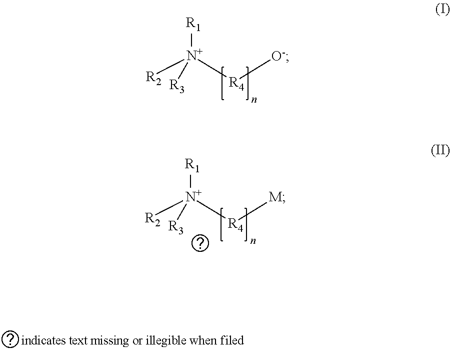
and
combinations thereof; and
wherein:
R₁ is a C₁-C₄ hydrocarbyl group,
n is an integer from 1 to 100, and
M is a hydrogen, or a metal ion; and
wherein the amount of the treating additive within the treated stream ranges from about 1 ppm to about 50,000 ppm.

18. The treated stream of claim 17, wherein the sulfur species is selected from the group consisting of hydrogen sulfide, mercaptans, polysulfides, and combinations thereof.

19. The treated stream of claim 17, wherein the metal ion is selected from the group consisting of Li, Na, K, and combinations thereof.

20. The treating additive of claim 17, wherein the compound (II) is selected from the group consisting of: 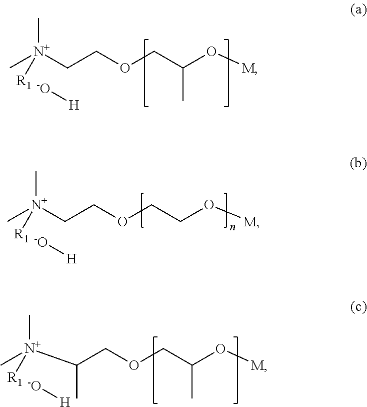
and
combinations thereof.
